(19) United States
(12) Patent Application Publication
(10) Pub. No.: US 2012/0095527 A1
(43) Pub. Date: Apr. 19, 2012

(54) SELF EXPANDABLE MIDDLE EAR IMPLANT FOR TREATING HEARING RELATED DISORDERS

(75) Inventors: Michael Ariel Vardi, Herzliya (IL); Alon Shalev, Raanana (IL)

(73) Assignee: ESTIMME LTD., Tel Aviv (IL)

(21) Appl. No.: 13/258,296

(22) PCT Filed: Jun. 3, 2010
(86) PCT No.: PCT/IL10/00441

§ 371 Date: Sep. 21, 2011

Related U.S. Application Data
(60) Provisional application No. 61/221,259, filed on Jun. 29, 2009.

(30) Foreign Application Priority Data
Jun. 8, 2009 (IL) 199243

(51) Int. Cl. A61F 11/04; A61N 1/05

(52) U.S. Cl. 607/57; 607/137

(57) ABSTRACT

The invention provides an electrode, and a minimally-invasive auditory implant system employing the electrode, for treating hearing disorders by electrically stimulating tissues in the middle ear. The electrode employs a structure which switches between narrow and spread shapes, facilitating the electrode insertion into the site, securing the electrode against vibration or permanent movement, and optimizing the current density.

### FIELD OF THE INVENTION

This invention relates generally to a system and method for treatment of hearing disorders and more particularly to a system which includes a middle ear effecting electrode for applying electrical signals having an arbitrary waveform to the cochlea. More specifically, this invention relates to a system that self expandably anchors into and optionally is easily retrievable from a region of the middle ear. Furthermore, this invention relates to electrodes for relatively low-invasiveness application of electrical signals to the cochlea, in the vicinity of the round window niche.

### BACKGROUND OF THE INVENTION

There are a number of hearing disorders which cause a great deal of suffering to mankind and various attempts have been made to relieve them. These disorders include: hearing loss in general, sensoryneural and conduction hearing loss in particular, mixed hearing loss, tinnitus, Meniere's disease and vertigo.

Contemporary interventional approaches to addressing such disorders may include providing electrical signals—whether inhibitory or excitatory, subthreshold or suprathreshold—to the nerve cells that convey the auditory signal from the inner ear into the brain, also known as the cochlear nerve.

Such delivery of electrical signal to the cochlear nerve may be accomplished via establishing a direct electrical interface with the cochlear nerve endings that reside in the inner ear, such as being done in an inner ear cochlear implant. Non specific electrical stimulation of the cochlear nerve may also be achieved via application of electricity in the middle ear, via an electrode that is in galvanic contact with the fenestra rotunda (the round window), with the promontorium or with adjacent tissue in the middle ear.

There is some body of prior art pertaining to the concept of applying electrical stimuli to the cochlear nerve in a minimally invasive fashion, via providing the electrical interface in the middle ear, at the promontory and/or adjacent one of the membranous windows. Nonetheless, a need exists to secure components of such a middle ear system, so that they will neither migrate nor undergo any significant vibration, during daily activities that a patient treated with such a system undergoes.

There have been attempts to address the issue of providing long term attachment between a middle ear electrode and the tissue neighboring the round window. For example, Kuzma (U.S. Pat. No. 4,809,712 and US patent application 20070213787) describes soft ball electrodes that are made of a conductive wire, and that are adapted for adjustment and customization into the round window niche.

Maltan et. al have described (US patent application 20070021804) an electrical stimulation system adapted to be implanted in a surgically created bony recess in the middle ear, wherein the electrode is placed in such a manner so as to stimulate the auditory system in order to affect tinnitus.

Rubinstein et. al. described the Electrical Suppression of Tinnitus with High-Rate Pulse Trains, achieved by a transient placement of a rod-like electrode on the promontorium, wherein the electrical stimulator was located outside the patient's body, and electrical leads were connecting between the electrical stimulator and the electrode, via a surgically created opening in the tympanic membrane.

In a previous patent application by one of the current applicants (US patent application 2009037689), an auditory implant system for treating a hearing disorder is disclosed, which is shaped and adapted for disposition in a Eustachian tube in the proximity to the round window.

The Eustachian tube (also often referred to as the auditory tube) is a collapsible passage that links the (naso)pharynx to the middle ear. In adults the Eustachian tube is approximately 35 mm long, and extends from the anterior wall of the middle ear to the lateral wall of the nasopharynx, approximately at the level of the inferior nasal concha. A portion of the tube proximal to the middle ear is made of bone; the rest is composed of cartilage and raises a tubal elevation, the torus tubarius, in the nasopharynx where it opens. The Eustachian tube represents a much less invasive implantation route, compared to contemporary methods for placing cochlear electrodes directly in the middle ear—risking infection and possibly irreversible damage to sensitive sensory and other neural structures.

Furthermore- the distal end of the Eustachian tube (i.e. its end that connects to the middle ear) may represent a natural cavity that is suitable for placement of a middle ear implant, not requiring any drilling or other trauma to bone. It is also very reasonable to have such a middle ear implant to be placed into the hypotympanum, utilizing it's natural concavity for long-term securing.

It is a long felt and unmet need therefore to provide a minimally invasive auditory implant system, including suitable electrodes, for treating a hearing disorder. It is therefore an object of this invention to provide the middle ear electrode for minimally invasive introduction into the vicinity of the round window, wherein said minimal invasiveness relates to each of i) conveying, positioning and deploying msaid electrode inside the middle ear, ii) contacting the fenestra rotunda or its close vicinity, iii) delivering electricity into the target ear aforementioned middle tissue, and iv) optionally retrieving the implant should such need arise.

It is another object of this invention to provide the middle ear electrode delivering sufficient electricity to cochlea with a reduced current density, compared to using, e.g., a needle electrode.

It is still another object of this invention to provide the middle ear electrode with reduced crossing profile, facilitating its placement through a small puncture in the eardrum (i.e. tympanic membrane) or through the Eustachian tube.

It is a further object of this invention to provide the middle ear electrode comprising a rotation mechanism enabling radial spreading of its distal parts, near to the target tissue.

It is a still further object of this invention to provide the middle ear electrode suitable for connecting with support structure located in the Eustachian tube.

Other objects and advantages of present invention will appear as description proceeds.

### SUMMARY OF THE INVENTION

The invention provides an electrode for delivering electricity in a target tissue in the vicinity of the round window (fenestra rotunda) niche in the middle ear; said electrode being defined by a longitudinal axis and having a proximal end and a distal end; said electrode comprising an elastic projection member for electrically interfacing said round window, the member disposed along said distal end; the member switching from radially narrowed shape to radially spread shape when contacting said round window, said spread shape reducing the average electrical current density at the round window, and said narrowed shape facilitating the electrode insertion into said niche; said proximal end being connected to a support structure located in the Eustachian tube. Said elastic projection member preferably comprises a plurality of elongated elastic projections for contacting said round window, the interface area of said projections in said spread shape being larger than in said narrowed shape. Provided is an electrode for delivering electricity in a target tissue in the vicinity of the round window niche in the middle ear, said electrode being defined by a longitudinal axis and having a proximal end and a distal end; said electrode comprising at least a portion of a generally cylindrical shell; said shell comprising a plurality of elongated elastic projections for contacting said round window, disposed along its said distal end; the projections being radially spread when being pushed along said axis against a solid plane, thereby increasing the contact area between said projections and said plane; said projections being made of an electrically conductive material. In a preferred embodiment of the invention, said electrode for delivering electricity in a target tissue in the vicinity of the round window niche in the middle ear is defined by a longitudinal axis, and has a proximal end and a distal end, while comprising at least a portion of a generally cylindrical shell, said shell comprising a plurality of elongated elastic projections for electrically interfacing said round window, disposed along its said distal end; the projections being able to assume a radially spread state and a radially narrowed state, wherein said radially spread state enables reducing the average current density to cochlea, and wherein said radially narrowed state reduces the crossing profile of said electrode, thereby facilitating its minimally invasive placement and retrieval; said projections being made of an electrically conductive material; the electrode being connectable at its proximal end to a support structure. Said projections preferably assume said radially spread state when longitudinally pressed in the distal to proximal direction. In one embodiment of the invention, said projections are mechanically coupled in at least a proximal coupling location and a distal coupling location; wherein adjoining said proximal coupling location to said distal coupling location results in said radially spread state, and wherein separating said proximal coupling location from said distal coupling location results in said radially narrowed state. Said radially spread or extended state enables to stabilize the electrode at the desired site, and further it enables to lower the electrical current density, as the overall current passes through an increased surface area, which lowers the risk of inadvertently denaturing proteins or otherwise damaging the tissues near the fenestra rotunda. In another embodiment of the invention, said projections are mechanically coupled in at least a proximal coupling circumference and a distal coupling circumference; wherein rotating said proximal coupling circumference with respect to said distal coupling circumference in a selected direction results in said radially spread state, and wherein rotating said proximal coupling circumference with respect to said distal coupling circumference opposite to said selected direction results in said radially narrowed state. Said elastic projection member may have the form selected from the group consisting of elongated rod projections, essentially spherical metal mesh, convex metal foil, a plurality of loops, helical winding, plurality of metal wire protrusions. In a preferred embodiment, the electrode according to the invention comprises a hollow shaft oriented along its longitudinal axis, and a second shaft thrust in said hollow shaft, wherein said second shaft is attached to said elastic projection member. Said second shaft may be slideably coupled to said first shaft, wherein sliding of said second shaft in the distal-proximal direction results in radial narrowing of said elastic projection member, and wherein sliding of said second shaft in the proximal-distal direction results in radial spreading of said elastic projection member. Said second shaft may be, alternatively, rotationally coupled to said first shaft, wherein rotating of said second shaft in the clockwise and counter-clockwise direction result in radial narrowing or spreading of said elastic projection member. The term “spreading”, employed at this context, means an extension or protrusion of at least a part of said electrode in the direction perpendicular to said longitudinal axis. The electrode according to the invention may be attached at its proximal end to a support structure placed in the Eustachian tube or in the hypotympanum, which structure is adopted for conveying the electrode from the Eustachian tube or from the hypotympanum, to the proximity of the fenestra rotunda, and for being stably anchored in the Eustachian tube or in the hypotympanum. In one embodiment, the electrode according to the invention may be attached at its proximal end to a translation and rotation mechanism enabling reduced-invasiveness electrode relocation in said niche. The electrode according to the invention is advantageously used in treating a hearing problem comprising a condition selected from tinnitus, Meniere's disease, dizziness, otosclerosis, and conductive or sensorineural or mixed hearing loss.

The invention is directed to a minimally-invasive auditory implant system for implantation into a middle ear comprising at least one electrode as described above or an array of electrodes comprising at least one electrode as described above, said system comprising i) a pulse generator (PG); and ii) a self: expandable support structure, adapted for anchoring in at least a portion of a hypotympanum of said middle ear, to which said electrode is mounted; wherein said support structure is adapted for transitioning between a compressed (conveying) configuration and a relaxed (anchoring) configuration, said configurations facilitating the conveyance or retrieval of said support structure in said at least a portion of hypotympanum and anchoring of said support, respectively, and wherein said compressed (conveying) configuration constitutes a spatially collapsed configuration, and wherein said relaxed (anchoring) configuration constitutes a spatially expanded configuration, and wherein said electrode is a cochlear effecting electrode (CEE) adapted for disposition in said middle ear in proximity to an associated fenestra rotunda and secured against vibration and permanent movement, said vibration or permanent movement, or other undesired movements, being possibly caused during daily activities of a treated subject. In a preferred embodiment of the invention, said PG is mounted to said support structure in said implant system. Said support structure in the system of the invention comprises, in one aspect, a generally convex mesh. Said support structure in the system of the invention comprises, in another aspect, a super-elastic metal. Said super-elastic metal may comprise nitinol or elginoy. The system of the invention preferably further comprises a delivery apparatus for releasably deploying said support structure within said at least a portion of hypotympanum, thereby actuating transitioning of said support structure from said compressed configuration to said relaxed configuration. Said delivery apparatus may comprise an endoscopic visualization channel. Said support structure in the system of the invention may be adapted for endoluminal retrieval following said transition from said relaxed configuration to said compressed configuration. In a preferred embodiment, said support structure comprises a plurality of retrieval handles, said handles adapted to engage a retrieval apparatus, said retrieval apparatus provided with means of transitioning said support structure from its said relaxed configuration to its said compressed configuration and its subsequent disposition into a generally elongated sheath. The system may comprise a return electrode, comprised in said support structure. Said system may comprise an extension arm, said extension arm having a proximal end and a distal end; said proximal end of extension arm being coupled to said support structure; said distal end of extension are being coupled to at least a portion of said array of electrodes. The above said generally convex mesh preferably comprises a portion of a generally spherical shell or a portion of a generally ovoid shell. In a preferred embodiment of the system according to the invention, said PG is mounted to said support structure in its concavity. In another preferred embodiment, said CEE is mounted to said support structure in its concavity. In one embodiment, the system according to the invention comprises an extension arm having a proximal end and a distal end, said proximal end of extension arm being coupled to said support structure, said distal end of extension being coupled to at least a portion of said array of electrodes. Said extension arm may be mounted to said support structure in its concavity. Said convex mesh may comprise radial support elements and generally circular support elements, wherein said circular support elements may be concentrically disposed therebetween. Said radial support elements may be connected with said circular support elements. Said circular support elements may be radially compressible. Said PG may be mounted to said support structure while comprising snap-fitting or screwing.

In one aspect of the invention, the implant system comprises one or more items selected from the group consisting of retrieval handles adapted to engage said retrieval apparatus, a return electrode, an extension arm coupled to said support structure and to said electrode. Said system preferably further comprises an extension arm, said extension arm having a proximal end and a distal end; said proximal end of extension arm being coupled to said support structure; said distal end of extension are being coupled to at least a portion of said array of electrodes.

### BRIEF DESCRIPTION OF THE DRAWINGS

The above and other characteristics and advantages of the invention will be more readily apparent through the following examples, and with reference to the appended drawings, wherein:

FIG. 1. schematically depicts the middle ear electrode (100), its longitudinal axis (101), its proximal end (102) and a distal end (103), a generally cylindrical shell (104) and a plurality of elongated projections (105) disposed along the distal (106) end of the cylindrical shell (104). The figure also depicts a lateral recess (107) that is adapted to engage a distal end of an extension arm;

FIG. 2. schematically depicts the middle ear electrode (120), comprising a generally convex, metal mesh (121), comprising haxagon and pentagon cells—much like a soccer ball—having a proximal end (122) and a distal end (123), at which one apex is disposed. An electrically insulated interface member (124) is disposed at the proximal end (122) of the electrode;

FIG. 3. schematically depicts the middle ear electrode (130), comprising a generally convex metal mesh (131), comprising a woven super-elastic metal wire, having a proximal end (132) and a distal end (133), at which one apex is disposed; an electrically insulated interface member (134) is disposed at the proximal end (132) of the electrode;

FIG. 4. schematically depicts the middle ear electrode (140), comprising a first elongated shaft member (141), a second elongated shaft member (142) and an electrode-head member (143), wherein the first (141) and second (142) elongated shafts being slideably coupled therebetween; the distal end of second shaft (144) is longitudinally coupled with the proximal end of the electrode-head member (145); furthermore, the distal end of the electrode-head member (146) is secureably attached to the distal end of the first shaft (147); the electrode-head member comprises a plurality of longitudinal conducting members (148) that are connected therebetween at least in the vicinity of the distal end of the electrode-head member—elastic projection member in the form of several loops (146); pulling the first shaft (141) proximally with regard to the second shaft causes said longitudinal conducting members to collapse radially outward; the figure further depicts an internal elastic member (149) that is adapted to prevent the longitudinal conducting members (148) from collapsing radially inward, while the first shaft (141) is being pulled proximally with regard to the second shaft (142);

FIG. 5. schematically depicts the middle ear electrode (150) being defined by a distally convex metal foil (151), proximally formed into generally longitudinal processes (152), a distal apex (153), and a ball-shaped electrically insulated interface member (154) that is disposed at the concavity of said metal foil; in selected embodiments of the present invention, the ball-shaped electrically insulated interface member (154) is made of a soft, compressible, or otherwise deformable material, so as to allow its conformity with the shape of the round window niche;

FIG. 6. schematically depicts the middle ear electrode (160), comprising a conductive wire having a first end (161) and a second end (162), a proximal portion (163) and a distal portion (164); the electrode comprises helical windings. The helical windings having a proximal (165) end and a distal end (166); the helical windings further being characterized by a linear length (167) and by an external diameter (168). The proximal end of the helical windings (165) is directed towards said first end of the conductive wire (161); the distal end of the helical windings (166) is directed towards the second end of the wire (162); the proximal portion the conductive wire (163) being defined as said wire disposed between said first end of said conductive wire and said proximal end of helical windings; said distal portion of conductive wire being defined as said wire disposed between said distal end of helical windings and said second end of said conductive wire;

FIG. 7. depicts a similar device as in FIG. 6, wherein its helical windings have a gradually increasing diameter in the distal direction;

FIGS. 8. (8a and 8b) schematically depict an electrode for the round window niche in the middle ear, comprising a static shaft (180), a rotating shaft (181), and a plurality of metal wire elements (191); A disc-shaped end element (184) is mechanically coupled to the distal end of the rotating shaft (181); the static shaft (180) and the rotating shaft (181) both comprising a proximal end (182 and 183, respectively) and a distal end (186 and 187, respectively); the static shaft (180) and the rotating shaft (181) are generally elongated and in this preferred embodiment—concentric to each other; FIG. 8a depicts the plurality of metal wire elements (191) in a radially minimized state, so as to facilitate introduction of the distal end of the electrode into the round window niche; FIG. 8b depicts the plurality of metal wire elements (191) in a radially expanded state, which is required for long-term fixation of the distal end of the electrode in the round window niche;

FIG. 9. depicts an electrode adapter (200) with a first and a second via holes (201 and 202 respectively), each said via hole (201 and 202 respectively) having a generally longitudinal cross section; the first via hole (201) has two ends (203 and 204, respectively); the second via hole (202) has two ends (205 and 206, respectively);

FIG. 10. depicts an electrode translation and rotation mechanism, comprising of a first and a second generally longitudinal guiding members (211 and 212, respectively) and a slideably coupled electrode adapter (200); the guiding members (211 and 212) comprise a plurality of lateral protrusions (collectively denoted as 213) that pass through the first and a second via holes in the electrode adapter (200); this figure depicts the guiding members (211 and 212, respectively) placed in contralateral ends of each via hole; the figure further depicts a generally tubular structural member (220) that is adapted to affix said translation and rotation mechanism to a generally hollow cavity in a mammalian body;

FIG. 11. depicts a similar electrode translation and rotation mechanism as in FIG. 10, however, the electrode adapter (200) is now shown to be longitudinally displaced along the longitudinal guiding members (211 and 212, respectively) and also clockwise rotated, compared to its position in FIG. 10. This figure depicts the guiding members (211 and 212, respectively) placed in ipsilateral ends of each via hole; the figure further depicts a generally tubular structural member (220) that is adapted to affix said translation and rotation mechanism to a generally hollow cavity in a mammalian body;

FIG. 12. schematically depicts a minimally invasive auditory implant, showing a pulse generator (310), a cochlear effecting electrode (CEE, 311) and an extension arm (312);

FIG. 13. schematically depicts a minimally invasive auditory implant, showing a pulse generator (310), a cochlear effecting electrode (CEE, 311), an extension arm (312) and a self-expandable support structure (313) in its relaxed state, which is not mounted with the aforementioned system components;

FIG. 14. schematically depicts another geometrical variant of minimally invasive auditory implant, showing a pulse generator (310), a cochlear effecting electrode (CEE, 311), an extension arm (312) and a self-expandable support structure (313) in its relaxed state, which is screw-mountable with the pulse generator (310);

FIG. 15. schematically depicts the minimally-invasive auditory implant system as in FIG. 13, wherein all components are assembled thereto. FIG. 15a depicts the implant system in its relaxed state. FIG. 15b depicts the implant system in its compressed state;

FIG. 16. schematically depicts the minimally-invasive auditory implant system as in FIG. 14, wherein all components are assembled thereto. FIG. 16a depicts the implant system in its relaxed state. FIG. 16b depicts the implant system in its compressed state;

FIG. 17. schematically depicts a minimally-invasive auditory implant system similar to one in FIG. 14, wherein all components are assembled thereto. The left hand side of the figure depicts the implant system in its relaxed state, while the right hand side of the figure depicts the implant system in its compressed state;

FIG. 18. schematically depicts another minimally-invasive auditory implant system as in FIG. 14, wherein all components are assembled thereto. The left hand side of the figure again depicts the implant system in its relaxed state, while the right hand side of the figure depicts the implant system in its compressed state; and

FIG. 19. schematically depicts a human middle ear (407). The figure depicts the oval window (400), the round window (401), the stapes (402), the malleus (403) and the incus (404). The figure also depicts the tympanic membrane (405), the medial portion of the external auditory meatus (406), the opening into the middle ear of the Eustachian tube (408) and the temporal bone (409). The figure also schematically depicts a minimally invasive auditory implant, showing a pulse generator (310), a cochlear effecting electrode (CEE, 311) located near the round window (301), an extension arm (312) and a self-expandable support structure (313) in its relaxed state, positioned in the hypotympanum.

### DESCRIPTION OF PREFERRED EMBODIMENTS

It has been found that the middle ear electrode may be located near fenestra rotunda without too invasive steps while securing its position, wherein relatively safely delivering electrical signals to cochlea, at low current densities, if employing an electrode according to the present invention. The electrode, for delivering electricity in a target tissue in the vicinity of the round window niche in the middle ear comprises at least a portion of a cylindrical shell and elongated elastic projections for contacting said round window, which projections switch from radially narrowed to radially spread form when being near said round window.

The invention provides the middle ear electrode for minimally invasive functioning near round window, wherein said minimal invasiveness relates to each of i) positioning said electrode inside the middle ear, ii) contacting fenestra rotunda, and iii) delivering electricity into the target middle-ear tissue. The electrode embodiments as appear in the invention, are capable to deliver to the cochlea a sufficient electrical current, while at a reduced current surface density. Switching between said narrowed and said spread states enables the reduction of the electrode crossing-profile, which facilitates its placement, preferably through either the external meatus (i.e. through an annulotomy or a puncture in the tympanic membrane) or via the Eustachian tube, from the nasopharynx. In various embodiments, the invention provides electrodes which are supported by a translational or rotational adjustments mechanisms, enabling radial spreading of its distal parts, near to the target tissue. The electrode is suitable to serve as a part in an auditory implant system for treating hearing disorders, advantageously connected with a support structure located in the middle ear—preferably in the distal end of the Eustachian tube or in the hypotympanum. The aforementioned system preferably comprises the support structure adapted for conveying an electrode from the Eustachian tube to the proximity of the fenestra rotunda, and for being anchored in said Eustachian tube or in the hypotympanum. In said system, the electrode is located stably in the middle ear, said stability being assured by attaching to said support structure, and possibly further enhanced by the electrode shape. Said support structure may function as a return electrode.

The present invention provides a minimally-invasive auditory implant system for implantation into a middle ear; the system comprising an array of electrodes, a pulse generator (PG), and a self-expandable support structure that is adapted for anchoring in at least a portion of a hypotympanum of the middle ear, to which at least a portion of said array is mounted; wherein said support structure is adapted for transitioning between a compressed (conveying) configuration and a relaxed (anchoring) configuration, said configurations respectively facilitating the conveyance and anchoring of said support structure in said at least a portion of hypotympanum, and wherein the compressed (conveying) configuration constitutes a spatially collapsed configuration, and wherein the relaxed (anchoring) configuration constitutes a spatially expanded configuration, and wherein the at least one electrode in said array is a cochlear effecting electrode (CEE) that is adapted for disposition in proximity to an associated fenestra rotunda.

In a preferred embodiment, the PG according to the invention is mounted to the support structure.

In a preferred embodiment, the support structure according to the invention comprises a generally convex mesh, which is intended to comply to the concave shape of the middle ear cavity—and specifically, the hypotympanum. In particular embodiments, the convex mesh may be a generally spherical shell, or an ovoid shell.

In a preferred embodiment, the support structure according to the invention comprises a super-elastic metal—such as nitinol, or elginoy—in order to allow for easy implantation of the implant in the middle ear, as well as easy retrieval thereof.

In a preferred embodiment, the minimally-invasive auditory implant system according to the invention is adjoined by a delivery apparatus for releasably deploying the support structure according to the invention within at least a portion of hypotympanum, thereby actuating transitioning of the support structure according to the invention from its compressed configuration to its relaxed configuration.

In a preferred embodiment, the delivery apparatus according to the invention further comprises an endoscopic visualization channel, so as to allow controlled and accurate deployment of the support structure according to the invention.

In another preferred embodiment, the support structure according to the invention is adapted for endoluminal retrieval following its transition from its relaxed configuration to its compressed configuration.

In yet another preferred embodiment, the support structure according to the invention further comprises a plurality of retrieval handles that are adapted to engage a retrieval apparatus. The retrieval apparatus is further provided with means of transitioning the support structure from its relaxed configuration to its compressed configuration and its subsequent disposition into a generally elongated sheath.

In a preferred embodiment, the minimally-invasive auditory implant system for implantation into a middle ear according to the invention additionally comprises a return electrode.

In yet another preferred embodiment, the minimally-invasive auditory implant system for implantation into a middle ear according to the invention additionally comprises a return electrode.

In a preferred embodiment, the minimally-invasive auditory implant system for implantation into a middle ear according to the invention further comprises an extension arm; the extension arm having a proximal end and a distal end; the proximal end of the extension arm being coupled to the support structure according to the invention; the distal end of extension are being coupled to at least a portion of the array of electrodes according to the invention.

In yet another preferred embodiment, the extension arm, according to the invention, having a proximal end and a distal end; the proximal end of extension arm being coupled to said support structure; the distal end of extension are being coupled to at least a portion of the array of electrodes, according to the invention. In particular embodiments, the extension arm is mounted to the support structure, according to the invention, in its concavity.

In a preferred embodiment, the PG according to the invention, is mounted to the support structure, according to the invention, in its concavity.

In a preferred embodiment, the CEE according to the invention, is mounted to the support structure, according to the invention, in its concavity.

In yet another preferred embodiment, convex mesh, according to the invention, comprises of radial support elements and generally circular support elements. In particular embodiments, the abovementioned circular support elements are concentrically disposed therebetween. In yet another particular embodiments, abovementioned radial support elements are connected with the abovementioned circular support elements. In yet another particular embodiments, the circular support elements are radially compressible.

The present invention provides an electrode for delivering electricity in a target tissue in the vicinity of the round window niche in the middle ear; said electrode being defined by a longitudinal axis and having a proximal end and a distal end; said electrode comprising an elastic projection member for electrically interfacing said round window, the member disposed along said distal end; the member switching from radially narrowed shape to radially spread shape when contacting said round window, said spread shape reducing the electrical current density to cochlea, and said narrowed shape facilitating the electrode insertion into said niche; said proximal end being connected to a support structure located in the Eustachian tube, or in the hypotympanum. Said elastic projection member may have the form of a plurality of elongated elastic projections for contacting said round window, the interface area of said projections in said spread shape being larger than in said narrowed shape, which ensures significantly reduced invasiveness of the electrode use, addressing both mechanical and electrical aspects. Said electrode preferably comprises at least a portion of a generally cylindrical shell; said shell comprising a plurality of elongated elastic projections for contacting said round window, disposed along its said distal end; the projections being radially spread when desired, thereby increasing the contact area between said projections and said plane; said projections being made of an electrically conductive material. In one aspect of the invention, said projections are radially spread when being pushed along said axis against a solid plane; such a plane may be represented by said round window, or a promontorium. In other aspect of the invention, said projections are radially spread when pushing a spreading mechanism in the proximal-distal direction, wherein said mechanism may comprise, for example, two concentric cylindrical shafts, inner and outer, slideably coupled, while the inner is connected with the elastic projections.

The electrode according to the invention preferably comprises at least a portion of a generally cylindrical shell, said shell comprising a plurality of elongated elastic projections for electrically interfacing said round window, disposed along its said distal end; the projections being able to assume a radially spread state and a radially narrowed state, wherein said radially spread state enables reducing the average current density to cochlea, and wherein said radially narrowed reduces the crossing profile of said electrode, thereby facilitating its minimally invasive placement and retrieval; said projections being made of an electrically conductive material; the electrode being connected at its proximal end to a support structure. Said projections assume said radially spread state when longitudinally pressed in the distal to proximal direction. Said projections are, in one embodiment of the invention, mechanically coupled in at least a proximal coupling location and a distal coupling location; wherein adjoining said proximal coupling location to said distal coupling location results in said radially spread state, and wherein separating said proximal coupling location from said distal coupling location results in said radially narrowed state. Said projections may be mechanically coupled in at least a proximal coupling circumference and a distal coupling circumference, wherein rotating said proximal coupling circumference with respect to said distal coupling circumference in a selected direction results in said radially spread state, and wherein rotating said proximal coupling circumference with respect to said distal coupling circumference opposite to said selected direction results in said radially narrowed state. Said elastic projection member of the electrode according to the invention has preferably a form selected from the group consisting of elongated rod projections, essentially spherical metal mesh, convex metal foil, a plurality of loops, helical winding, and plurality of metal wire protrusions. Such protrusions may have the form of regularly arranged loops or rods; the protrusions may have the form of randomly arranged loops, or fiber ball, a plurality of elongated projections of other forms. The protrusions may be arranged in a net-like surface, or in a foil-like surface, and may comprise a metal fiber, wire, or foil.

In other preferred embodiment, the electrode according to the invention comprises a hollow shaft oriented along said longitudinal axis of the electrode, and a second shaft thrust in said hollow shaft, wherein said second shaft is attached to said the elastic projection member. Said second shaft is slideably coupled to said first shaft, wherein sliding of said second shaft in the distal-proximal direction results in radial narrowing of said elastic projection member, and wherein sliding of said second shaft in the proximal-distal direction results in radial spreading of said elastic projection member. In another embodiment, said second shaft is rotationally coupled to said first shaft, wherein rotating of said second shaft in the clockwise and counter-clockwise direction results in radial narrowing or spreading of said elastic projection member. An electrode according to the invention is, in an important aspect, a part of an auditory implant system for treating hearing disorders, preferably attached at its proximal end to a support structure placed in the Eustachian tube or in the hypotympanum, which structure is adopted for conveying the electrode from the Eustachian tube to the proximity of the fenestra rotunda, and for being stably anchored in the Eustachian tube. An electrode according to the invention may be advantageously attached at its proximal end to a translation and rotation mechanism enabling non-invasive electrode relocation in said niche.

The invention aims at an electrode for use in treating a hearing problem comprising a condition selected from tinnitus, Meniere's disease, dizziness, otosclerosis, and conductive or sensorineural or mixed hearing loss.

The invention will be further described and illustrated in the following examples.

### EXAMPLES

### Example 1

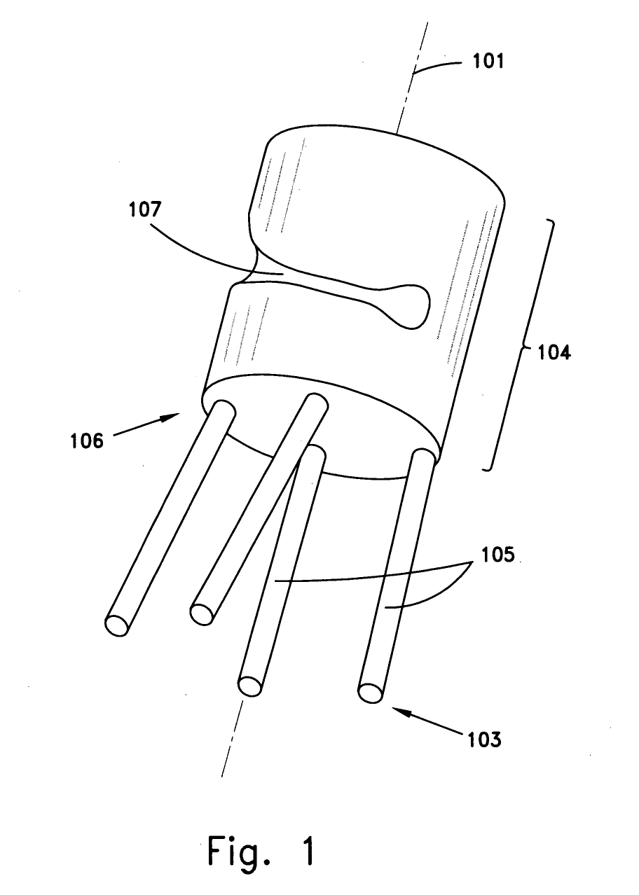

An embodiment of the invention is described herein (see FIG. 1 and the Description of the Drawings), namely an electrode for delivering electricity in a target tissue in the vicinity of the round window niche in the middle ear; said electrode being defined by a longitudinal axis and having a proximal end and a distal end; said electrode comprising at least a portion of a generally cylindrical shell; said shell comprising a plurality of elongated projections disposed along its said distal end; said electrode is made of an electrically conductive material. Said elongated projections are being generally aligned in parallel with said longitudinal axes. Said elongated projections are further being slightly outwardly radially protruding, whereas longitudinally compressing its said distal end against said target tissue results in said elongated projections being pushed radially outward into said target tissue. Said elongated projections may be further bent clockwise, whereas clockwise rotating its said distal end against said target tissue, while longitudinally compressing its said distal end against said target tissue, results in said elongated projections being pushed generally clockwise into said target tissue. Said clockwise direction may be replaced with counter-clockwise. The electrode may further comprise an electrically insulated interface member, said interface member being disposed at said proximal end of electrode. The electrode may further comprise an elongated engagement member having a proximal end and a distal end, said distal end of engagement member being coupled with said interface member; possibly, said distal end of engagement member being encapsulated within said interface member. Said engagement member may comprise an extension arm; said extension arm may comprise a metal. Said interface member may comprise a polymer mold. Said cylindrical shell may have a diameter preferably larger than about 2 millimeter. The electrode may further comprise a conductive lead; said lead being galvanically coupled to said electrode. Said electrode comprising an electrically insulated interface member disposed at said proximal end may further comprise a conductive lead galvanically coupled to said electrode; said lead being further coupled with said interface member while being adapted to affix said translation and rotation mechanism to a generally hollow cavity in a mammalian body.

### Example 2

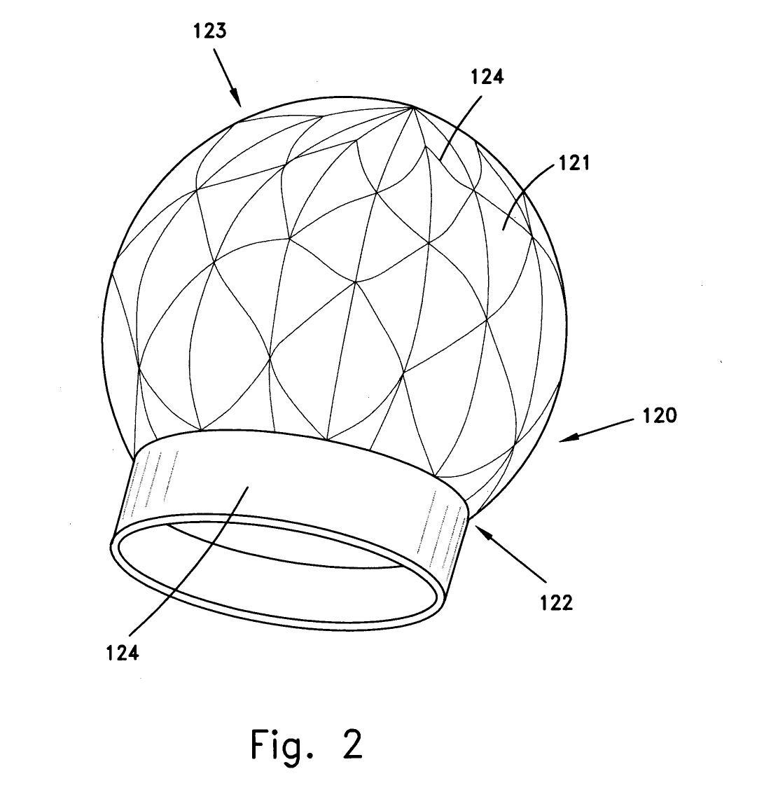

An embodiment of the invention is described herein (see FIG. 2 and the Description of the Drawings), namely an electrode for delivering electricity in a target tissue in the vicinity of the round window niche in the middle ear; said electrode being defined by a generally convex elastic metal mesh having a proximal end and a distal end. Said mesh comprises at least one apex being disposed at said distal end of electrode. The electrode further comprises an electrically insulated interface member, said interface member being disposed at said proximal end of electrode. The electrode further comprises an elongated engagement member having a proximal end and a distal end, said distal end of engagement member being coupled with said interface member; possibly, said distal end of engagement member being encapsulated within said interface member. Said engagement member preferably comprises an extension arm, which may comprises a metal. Said interface member may comprise a polymer mold. Said metal mesh is generally of ovoid or spherical in shape. Said metal mesh may be manufactured by cutting and folding a metal foil, or by winding a metal wire over a mandrel and subsequently removing said mandrel. Said metal mesh comprises voids that are adapted to allow in-growth of tissue there-through and thereby encapsulation of at least a portion of said distal end of electrode. The electrode may further comprise an electrically insulated interface member disposed at said proximal end of electrode, and a conductive lead being galvanically coupled to said metal mesh, said lead being further coupled with said interface member. The electrode may have a mesh comprising voids of different sizes. Said mesh may be braided from metal wires, or from a single metal wire, or said mesh is cut and shaped from a metallic foil.

### Example 3

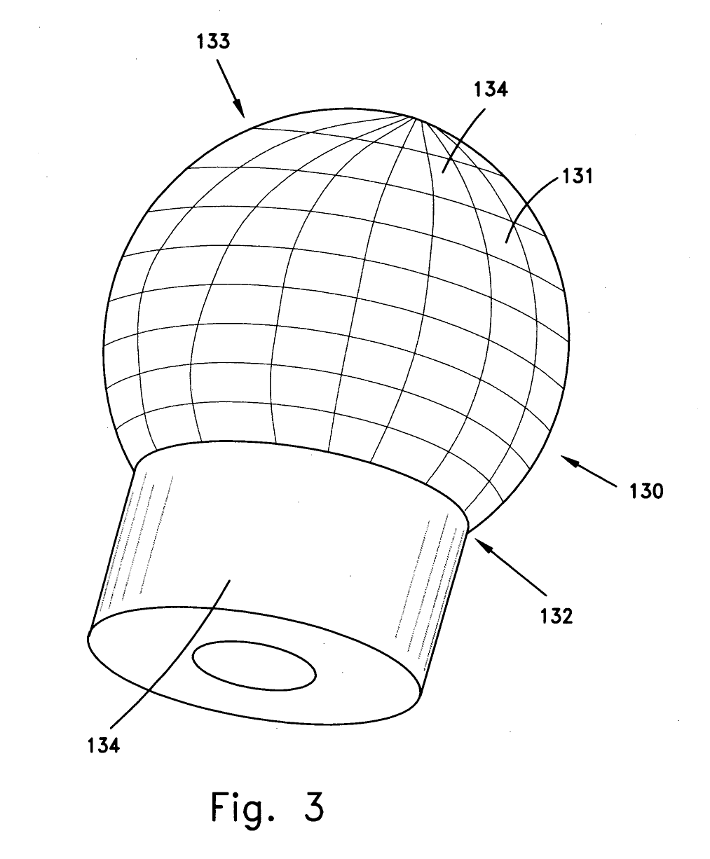

An embodiment of the invention is described herein (see FIG. 3 and the Description of the Drawings), namely an electrode for delivering electricity in a target tissue in the vicinity of the round window niche in the middle ear; said electrode comprising a conductive wire having a first end and a second end, a proximal portion and a distal portion, and a length; said electrode comprising helical windings at a selected position along its length; said helical windings having a proximal end and a distal end; said helical windings further being characterized by a linear length and by an external diameter; said proximal end of helical windings is directed towards said first end of conductive wire; said distal end of helical windings is directed towards said second end of wire; said proximal portion of conductive wire being defined as said wire disposed between said first end of said conductive wire and said proximal end of helical windings; said distal portion of conductive wire being defined as said wire disposed between said distal end of helical windings and said second end of said conductive wire. Said selected position is in proximity with said second end of conductive wire. Said second end of conductive wire is inserted into said distal end of helical windings. Said distal portion of conductive wire is generally disposed in parallel with said proximal portion of conductive wire. Said conductive wire in the electrode of this example may be made of multiple metallic filaments. Said conductive wire further comprises an external insulating cladding member; said cladding member being discontinued in at least a portion of said helical windings. The electrode further comprises an electrically insulated interface member, said interface member being disposed at said proximal end of helical windings; said electrode further comprises an elongated engagement member having a proximal end and a distal end, said distal end of engagement member being coupled with said interface member, wherein said distal end of engagement member may be encapsulated within said interface member; said engagement member comprises an extension arm; said extension arm comprises a metal; said interface member comprises a polymer mold. Said proximal end of helical windings and said distal end of helical windings, in the electrode of the Example, are attached therebetween so as to form a loop of helical windings. Said external diameter of helical windings is generally constant or is tapered down towards said distal end of helical windings.

### Example 4

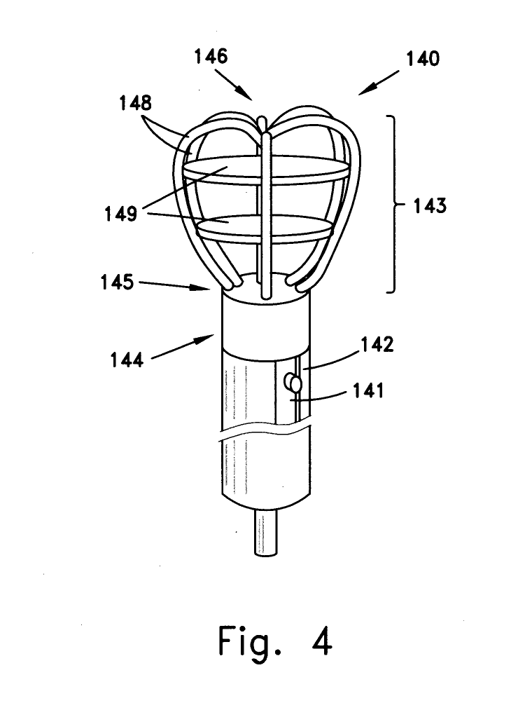

An embodiment of the invention is described herein (see FIG. 4 and the Description of the Drawings), namely an electrode for delivering electricity in a target tissue in the vicinity of the round window niche in the middle ear; said electrode comprising a first elongated shaft member, a second elongated shaft member and an elastic electrode-head member; said first and second elongated shafts being slideably coupled therebetween; said first shaft, second shaft and electrode-head member comprising a proximal end and a distal end; said distal end of second shaft being longitudinally coupled with said proximal end of electrode-head member; said distal end of electrode-head member being secureably attached to said distal end of first shaft; said electrode-head member comprising a plurality of longitudinal conducting members; said longitudinal conducting members being connected therebetween at least at said distal end of said electrode-head member; wherein pulling said first shaft proximally with regard to said second shaft causes said longitudinal conducting members to collapse radially outward. The electrode further comprises a locking member; said locking member adapted to securely affix said first shaft with regard to said second shaft. Said longitudinal conducting members may be further connected therebetween by a plurality of laterally expendable struts. Said electrode-head member may comprise a platinum alloy or a super elastic metal alloy. The electrode further comprises a conductive lead; said lead being galvanically coupled to said electrode-head member. Said electrode head member may further comprises an internal elastic member adapted to prevent said longitudinal conducting members from collapsing radially inward, while said first shaft is being pulled proximally with regard to said second shaft.

### Example 5

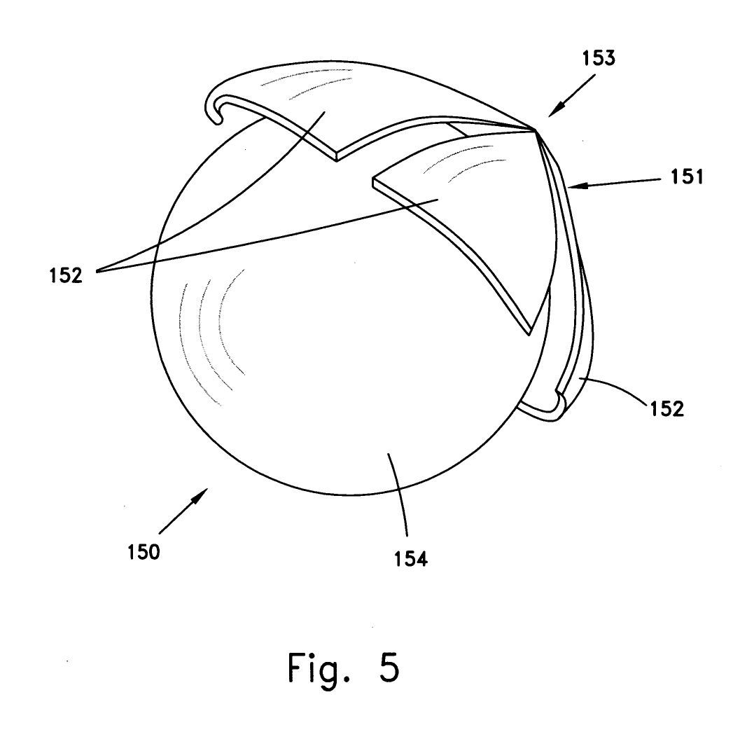

An embodiment of the invention is described herein (see FIG. 5 and the Description of the Drawings), namely an electrode for delivering electricity in a target tissue in the vicinity of the round window niche in the middle ear; said electrode being defined by a distally convex metal foil, proximally formed into generally longitudinal processes. The electrode comprises at least one distal apex, and an electrically insulated interface member being disposed at a concavity of said metal foil; the electrode may further comprise an elongated engagement member having a proximal end and a distal end, said distal end of engagement member being coupled with said interface member, said distal end of engagement member may be encapsulated within said interface member; said engagement member may comprise an extension arm; said extension arm may comprise a metal. Said electrode comprising an electrically insulated interface member being disposed at a concavity of said metal foil may comprise a polymer mold in said interface member. Said electrically insulated interface member being disposed at a concavity of said metal foil and said metal foil may jointly form a generally ovoid or spherical shape. The electrode may further comprise a conductive lead; said lead being galvanically coupled to said metal foil. Said electrode comprising an electrically insulated interface member being disposed at a concavity of said metal foil may further comprise a conductive lead galvanically coupled to said metal foil and to said interface member.

### Example 6

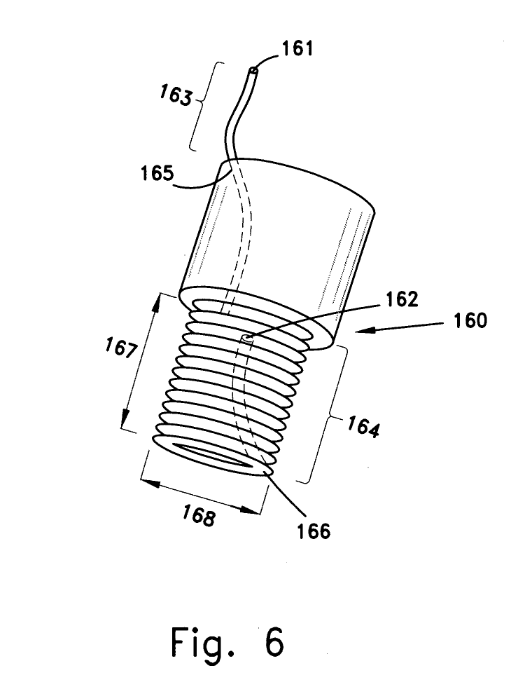

An embodiment of the invention is described herein (see FIG. 6 and the Description of the Drawings), namely a system for in-situ forming of a wire electrode in the round window niche in the middle ear; said system comprising a static shaft, a rotating shaft, a metal wire and a wire feeding lumen; said static shaft, rotating shaft and wire feeding lumen comprising a proximal end and a distal end and being generally elongated and parallel thereto; wherein said distal end of wire being connected to said distal end of rotating shaft, whereby rotating said rotating shaft while allowing said metal wire to advance distally through said feeding lumen creates winding of said metal wire around, or adjacent, said distal end of rotating shaft. The system further comprises a disengagement mechanism, adapted to disconnect said distal end of wire from said distal end of rotating shaft, thereby allowing for proximally withdrawing said rotating shaft; said connection between said distal end of wire and said distal end of rotating shaft comprises a fuse member, wherein said disengagement mechanism comprising an electrical-current driving mechanism adapted to burn said fuse member while not materially impacting said metal wire. Said static shaft may be a cylindrical tube, wherein said rotating shaft is being characterized by a generally circular cross section and being co-axially disposed inside said static shaft; said rotating shaft further comprising a cylindrical cross section; said rotating shaft may comprise wire feeding lumen. The space remaining inside said static shaft, and externally to said rotating shaft, preferably comprises said feeding lumen. Said static shaft and said rotating shaft are external to the system; said static shaft is a cylindrical tube; the internal volume of said static shaft comprising said feeding lumen.

### Example 7

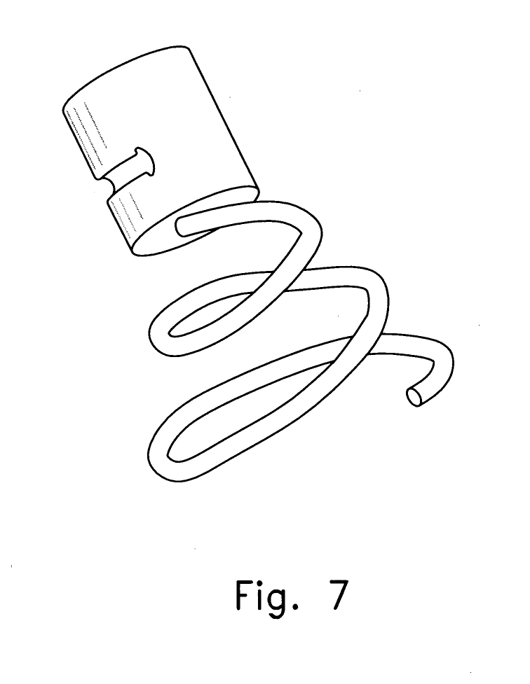

An embodiment of the invention is described herein (see FIG. 7 and the Description of the Drawings), namely an electrode for delivering electricity in a target tissue in the vicinity of the round window niche in the middle ear; said electrode comprising a plurality of generally elongated, galvanically connected metal wire member elements, being generally parallel aligned therebetween; said wire member element comprising a proximal end and a laterally bent distal end; said plurality of laterally bent distal ends defining a distal lateral electrode plane, said plane being characterized by an electrode footprint. Said electrode footprint assumes a diminished surface area when said plurality of wire member elements are approximated therebetween adjacent their respective distal ends; said electrode footprint assumes an enlarged surface area when said plurality of wire member elements are allowed to assumed their respective relaxed orientation. Alternatively, said electrode footprint assumes an enlarged surface area when said plurality of wire member elements are approximated therebetween adjacent their respective distal ends; said electrode footprint assumes a diminished surface area when said plurality of wire member elements are allowed to assumed their respective relaxed orientation. The electrode may further comprise a ring member, said ring member being placed upon said plurality of wire member elements and being capable of longitudinal displacement along said wire member elements; whereby displacement of said ring member adjacent distal end of said wire member causes said elements to be approximated therebetween.

### Example 8

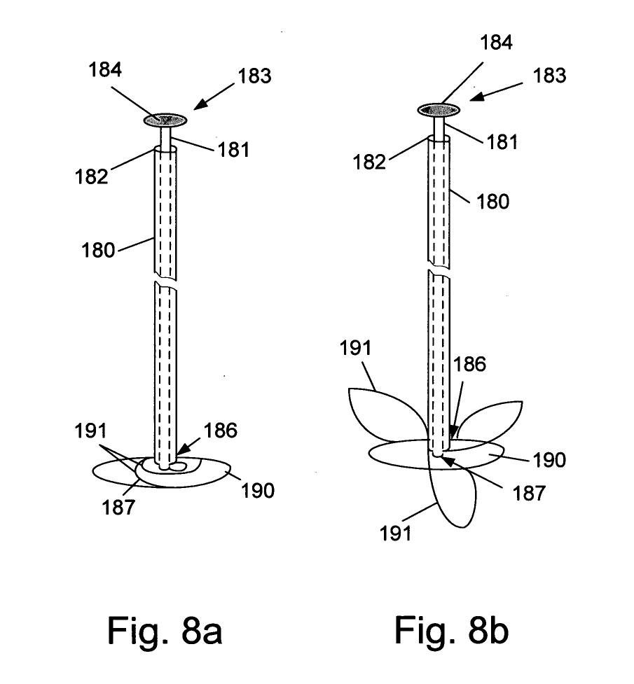

An embodiment of the invention is described herein (see FIG. 8 and the Description of the Drawings), namely an electrode for delivering electricity in a target tissue in the vicinity of the round window niche in the middle ear; said electrode comprising a static shaft, a rotating shaft, and a plurality of metal wire elements; said static shaft and rotating shaft comprising a proximal end and a distal end and being generally elongated and parallel therebetween; wherein said plurality of metal wire elements being disposed between locations along circumference of said distal end of rotating shaft and corresponding locations along circumference of said distal end of static shaft. The rotation of said rotating shaft relative to said static shaft is adapted to transform said metal wire elements from assuming a radially minimized state to assuming a radially expanded state, and vice versa. Said rotating shaft or said static shaft further comprise a generally circular plate, coupled to its distal end; said generally circular plate or disc being adapted to distally restrain said plurality of metal wire elements from protruding distally relative to said distal ends of either static shaft or said rotating shaft, during relative rotation of said shafts therebetween. Said static shaft and rotating shaft preferably comprise a circular rod and a cylindrical tube, respectively; said static shaft passing through said rotating shaft.

### Example 9

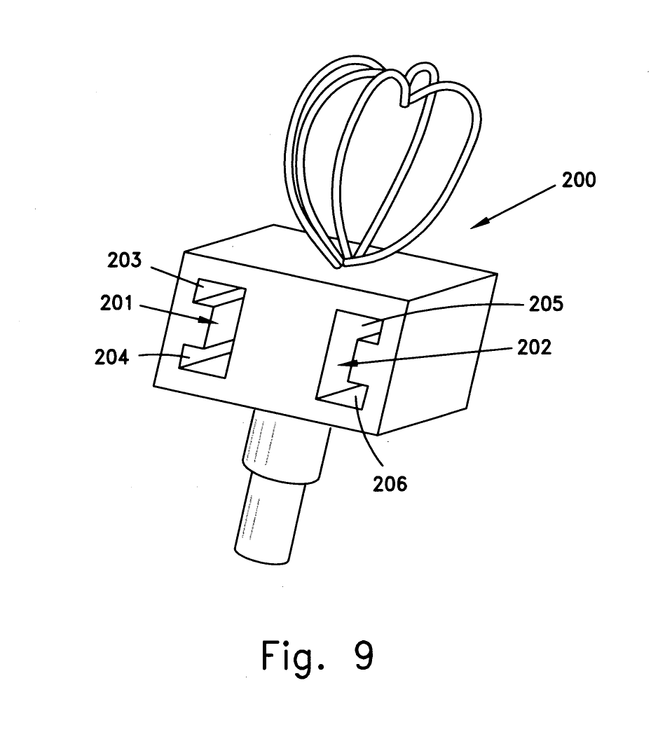

An embodiment of the invention is described herein (see FIG. 9 and the Description of the Drawings), namely an electrode translation and rotation mechanism, comprising a first and a second generally longitudinal guiding members and a slideably coupled electrode adapter; wherein said guiding members comprise a plurality of lateral protrusions, and wherein said electrode adapter comprises a first and a second via holes, each said via hole having a generally longitudinal cross section, said generally longitudinal cross section having at least two ends and at least one internal recess in the shape that matches said shape of lateral protrusion; wherein said electrode adapter may be translated along said first and second guiding members by either pulling or pushing said electrode adapter along the direction of said longitudinal members, whereas said lateral protrusions inhibit longitudinal movement of said electrode adapter with regard to said longitudinal members; and wherein said electrode adapter may be rotated with regard to said first and second guiding members by positioning each of said longitudinal members at either said ends of said via hole. Said generally longitudinal guiding members preferably comprise an elastic material, for example a super elastic metal. Said generally longitudinal guiding members are coupled to a radially expandable, generally tubular structural member; said structural member is adapted to affix said translation and rotation mechanism to a generally hollow cavity in a mammalian body.

### Examples 10-19

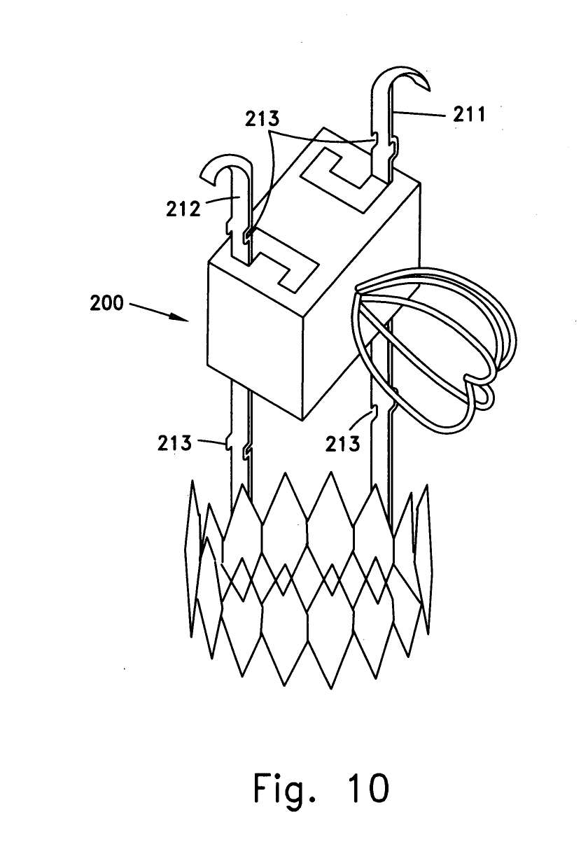

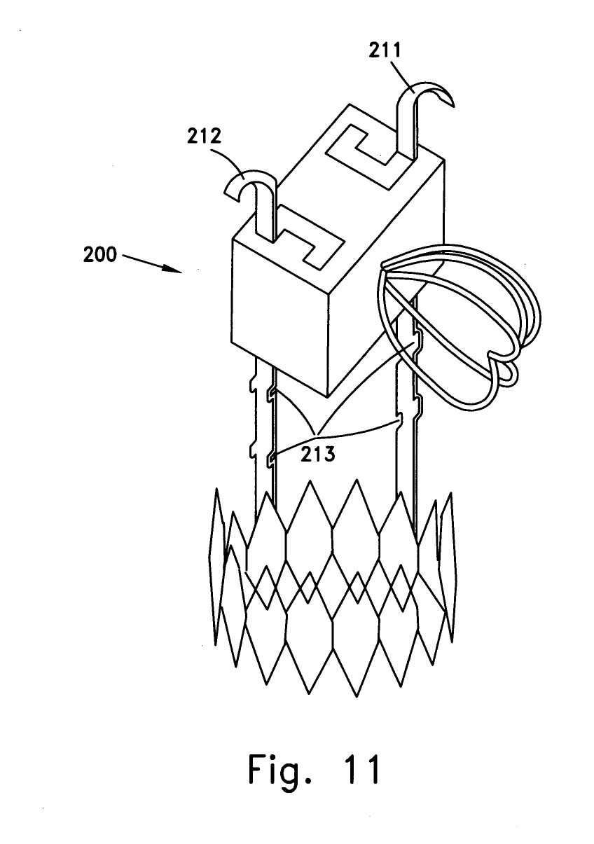

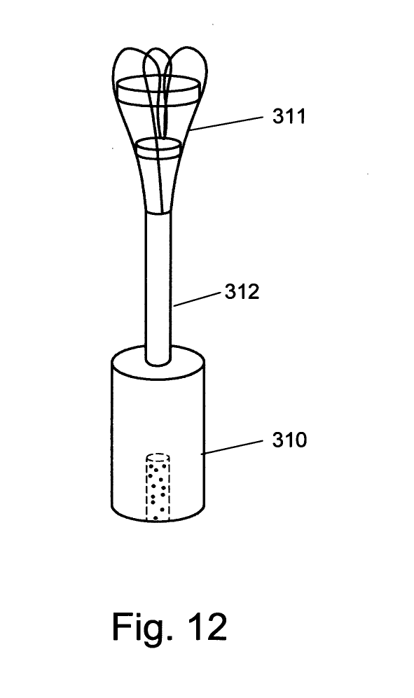

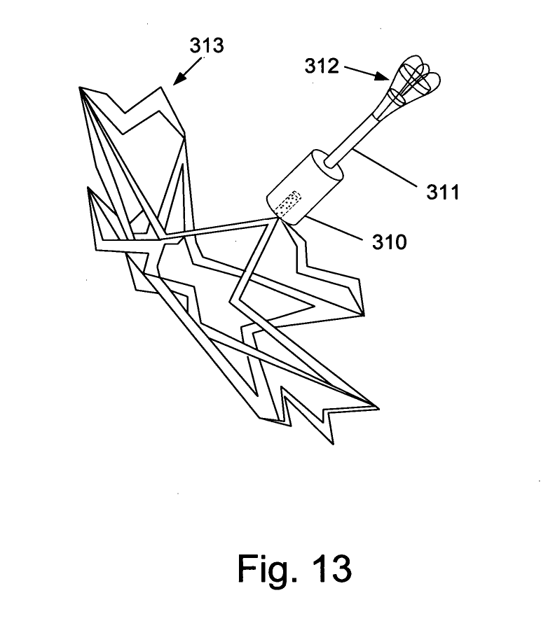

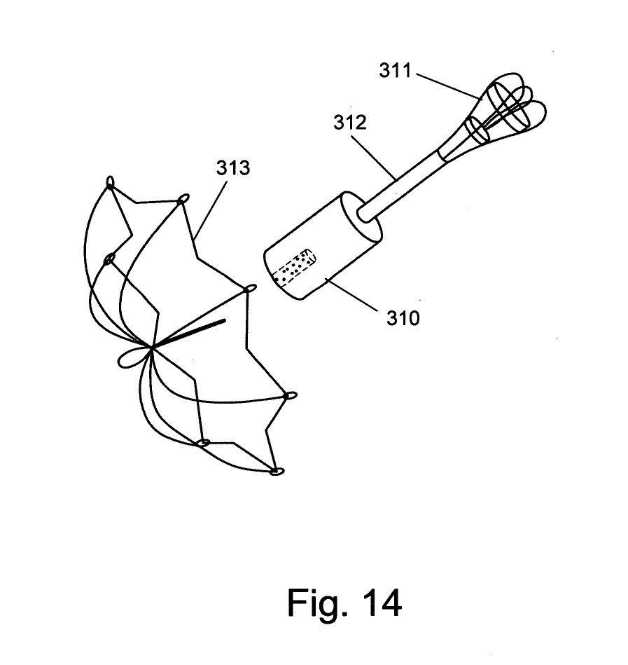

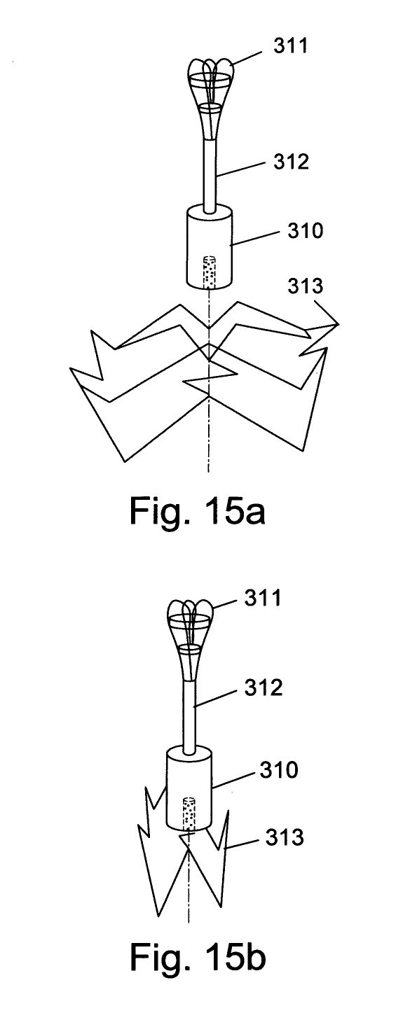

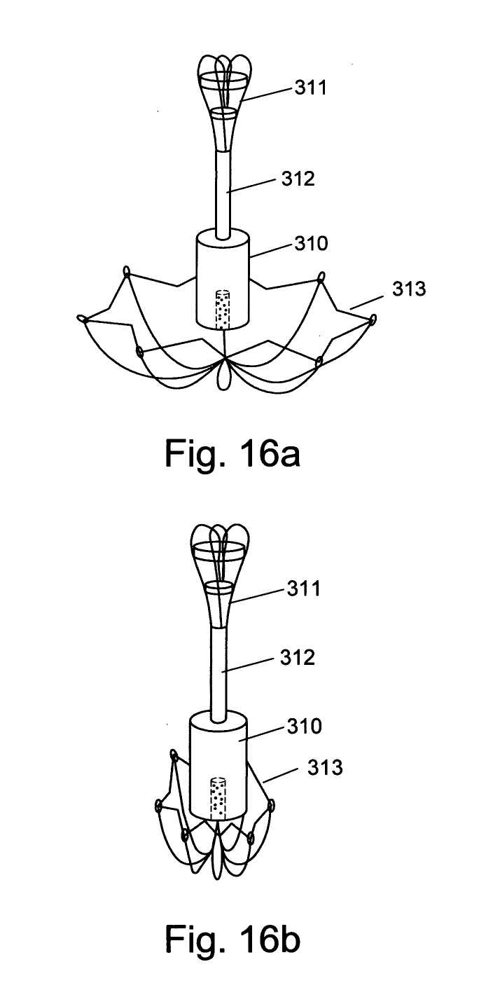

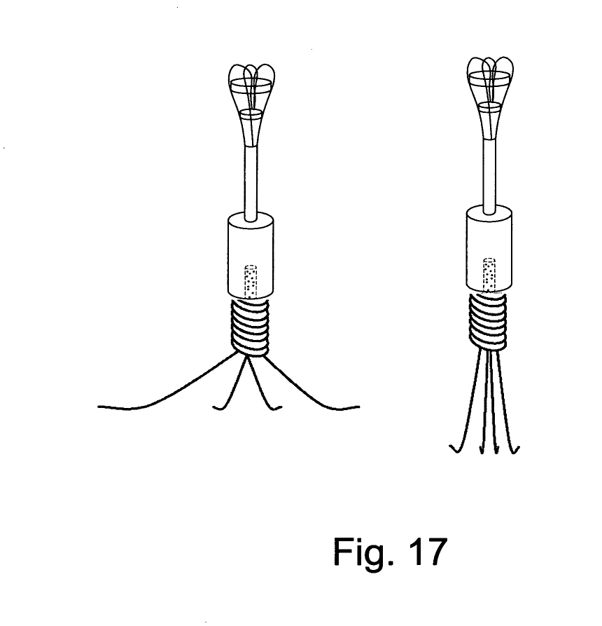

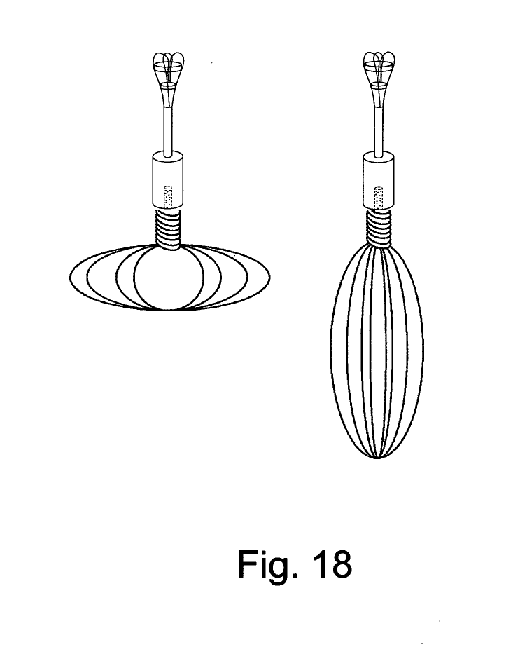

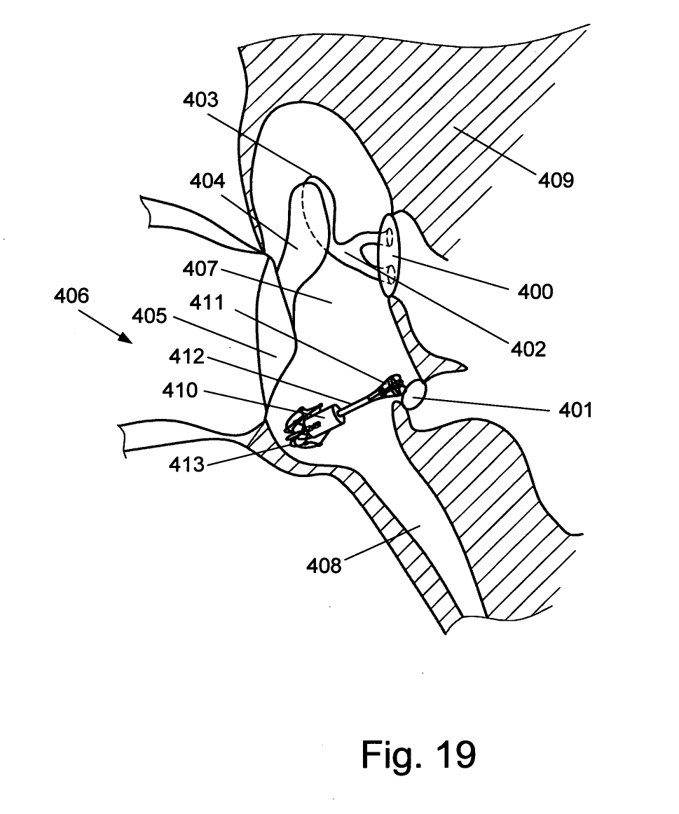

Additional embodiments of the invention are described herein (see FIG. 10 to FIG. 19 and the Description of the Drawings), namely electrode translation and rotation mechanisms, minimally invasive auditory implants, and systems comprising same.

While this invention has been described in terms of some specific examples, many modifications and variations are possible. It is therefore understood that within the scope of the appended claims, the invention may be realized otherwise than as specifically described.

1-24. (canceled)

25. An electrode for delivering electricity in a target tissue in the vicinity of the round window niche in the middle ear; said electrode being defined by a longitudinal axis and having a proximal end and a distal end, said electrode comprising an elastic projection member for electrically interfacing said round window, said member disposed along said distal end; said member switching from radially narrowed shape to radially spread shape when contacting said round window, when rotating or longitudally pressing said electrode, said spread shape reducing the electrical current density to cochlea and securing said electrode against vibration, and said narrowed shape facilitating the electrode insertion into said niche; said proximal end being connected to a support structure located in the Eustachian tube.

26. The electrode according to claim 25, wherein said elastic projection member comprises a plurality of elongated elastic projections for contacting said round window, the interface area of said projections in said spread shape being larger than in said narrowed shape.

27. The electrode according to claim 25, for delivering electricity in a target tissue in the vicinity of the round window niche in the middle ear; said electrode being defined by a longitudinal axis and having a proximal end and a distal end; said electrode comprising at least a portion of a generally cylindrical shell; said shell comprising a plurality of elongated elastic projections for contacting said round window, disposed along said distal end; the projections being radially spread when being pushed along said axis against a solid plane, thereby increasing the contact area between said projections and said plane; said projections being made of an electrically conductive material.

28. The electrode according to claim 25, for delivering electricity in a target tissue in the vicinity of the round window niche in the middle ear; said electrode being defined by a longitudinal axis and having a proximal end and a distal end; said electrode comprising at least a portion of a generally cylindrical shell; said shell comprising a plurality of elongated elastic projections for electrically interfacing said round window, disposed along said distal end; the projections being able to assume a radially spread state and a radially narrowed state, wherein said radially spread state enables reducing the average current density to cochlea, and wherein said radially narrowed state reduces the crossing profile of said electrode, thereby facilitating its minimally invasive placement and retrieval; said projections being made of an electrically conductive material; the electrode being connectable at its proximal end to a support structure.

29. The electrode according to claim 28, wherein said projections are mechanically coupled in at least a proximal coupling location and a distal coupling location; wherein adjoining said proximal coupling location to said distal coupling location results in said radially spread state, and wherein separating said proximal coupling location from said distal coupling location results in said radially narrowed state.

30. The electrode according to claim 28, wherein said projections are mechanically coupled in at least a proximal coupling circumference and a distal coupling circumference; wherein rotating said proximal coupling circumference with respect to said distal coupling circumference in a selected direction results in said radially spread state, and wherein rotating said proximal coupling circumference with respect to said distal coupling circumference opposite to said selected direction results in said radially narrowed state.

31. The electrode according to claim 25, wherein said elastic projection member has the form selected from the group consisting of elongated rod projections, essentially spherical metal mesh, convex metal foil, a plurality of loops, helical winding, plurality of metal wire protrusions.

32. The electrode according to claim 25, comprising a hollow shaft oriented along said longitudinal axis of the electrode, and a second shaft thrust in said hollow shaft and attached to said elastic projection member, wherein said second shaft is either
(a) slideably coupled to said first shaft, wherein sliding of said second shaft in the distal-proximal direction results in radial narrowing of said elastic projection member, and wherein sliding of said second shaft in the proximal-distal direction results in radial spreading of said elastic projection member, or
(b) rotationally coupled to said first shaft, wherein rotating of said second shaft in the clockwise and counter-clockwise direction results in radial narrowing and spreading, respectively, of said elastic projection member.

33. The electrode according to claim 25, being attached at its proximal end to a support structure placed in the Eustachian tube or in the hypotympanum, which structure is adopted for conveying the electrode from the Eustachian tube or from the hypotympanum to the proximity of the fenestra rotunda, and for being stably anchored in the Eustachian tube or in the hypotympanum, said electrode being further attached at its proximal end to a translation or rotation mechanism enabling reduced-invasiveness electrode relocation in said niche.

34. The electrode according to claim 25, for use in treating a hearing problem comprising a condition selected from tinnitus, Meniere's disease, dizziness, otosclerosis, and conductive or sensorineural or mixed hearing loss.

35. A minimally-invasive auditory implant system for implantation into a middle ear comprising an electrode according to claim 25, said system comprising
(i) a pulse generator (PG); and
(ii) a spatially-expandable support structure, adapted for anchoring in at least a portion of a hypotympanum of said middle ear, to which said electrode is mounted;
wherein said support structure is adapted for transitioning between a compressed (conveying) configuration and a relaxed (anchoring) configuration, said configurations facilitating the conveyance or retrieval of said support structure in said at least a portion of hypotympanum and anchoring of said support, respectively, and
wherein said compressed (conveying) configuration constitutes a spatially collapsed configuration, and wherein said relaxed (anchoring) configuration constitutes a spatially expanded configuration, and
wherein said electrode is a cochlear effecting electrode (CEE) adapted for disposition in said middle ear, in proximity to the associated fenestra rotunda and secured against vibration and permanent movement.

36. The system of claim 35, wherein said PG is mounted to said support structure.

37. The system of claim 35, wherein said support structure comprises a generally convex mesh or super-elastic metal comprising nitinol or elginoy.

38. The system of claim 35, further comprising a delivery apparatus for releaseably deploying said support structure within said at least a portion of hypotympanum, thereby actuating transitioning of said support structure from said compressed configuration to said relaxed configuration, said system further comprising one or more items selected from the group consisting of retrieval handles adapted to retrieve said support structure, a return electrode, endoscopic visualization channel, and an extension arm coupled to said support structure and to said electrode.

39. The system of claim 35, wherein said spatially-extendable support structure is a self-extendable support structure.
